(19) United States
(12) Patent Application Publication
(10) Pub. No.: US 2024/0158826 A1
(43) Pub. Date: May 16, 2024

(54) PREPARATION METHOD OF SYNTHETASE FOR CONTINUOUS MONITORING OF BLOOD GLUCOSE

(71) Applicants: Shaoda ZHANG, Guangdong (CN); Zhipeng Qiu, Guangdong (CN); Xingyu Zheng, Guangdong (CN)

(72) Inventors: Shaoda ZHANG, Guangdong (CN); Zhipeng Qiu, Guangdong (CN); Xingyu Zheng, Guangdong (CN)

(21) Appl. No.: 18/206,088

(22) Filed: Jun. 6, 2023

(30) Foreign Application Priority Data
Nov. 16, 2022 (CN) 202211432529.1

(51) Int. Cl. C12Q 1/00; C12N 9/12; C12N 11/14; C12Q 1/54

(52) U.S. Cl. CPC C12Q 1/006; C12N 9/1241; C12N 11/14; C12Q 1/54; C12Y 207/07027

(57) ABSTRACT

The present invention discloses a preparation method of a synthetase for continuous monitoring of blood glucose. The method includes the preparation steps of step S1: hydroxylation of a glass bead; step S2: silanization of the glass bead; step S3: bonding of the glass bead to glucose; step S4: synthesis of a polymer particle on the surface of the glass bead to form a glucose synthetase; and step S5: separation of the highly specific glucose synthetase. According to the preparation method of a synthetase for continuous monitoring of blood glucose provided by the present invention, the glass bead is bonded to the glucose by activating the surface of the glass bead, and then bonded to a polymer to form the glucose synthetase. The artificial synthetase prepared by the method has low sensitivity to temperature and pH value, and can be directly used in a blood glucose electrochemical sensor.

### BACKGROUND OF THE INVENTION

The present invention relates to the technical field of synthetases, and more specifically, relates to a preparation method of a synthetase for continuous monitoring of blood glucose.

Diabetes mellitus is a metabolic disorder syndrome caused by hypoinsulinism, insulin resistance and the like when genetic factors, immune factors and other pathogenic factors act on the body. During treatment of diabetic patients, it is very necessary to detect blood glucose of the patients. Bioelectrochemical sensors have the advantages of simplicity, convenience, low price, high sensitivity and the like, thus having been widely used in medical and health treatment, which play a major role in the detection of the blood glucose of the diabetic patients. Among the bioelectrochemical sensors, a glucose bioelectrochemical sensor is usually used for monitoring the blood glucose. Based on testing principles, the glucose bioelectrochemical sensor has a variety of methods, including an oxidase method, a spectral analysis method, a fluorescence detection method and the like. At present, a glucose oxidase method is the most mature technology with highest detection precision. That is to say, a glucose oxidase is fixed on an electrode, the electrode is placed in a testing solution, and then a current signal of a reaction of glucose and the glucose oxidase in the testing solution is detected, so as to determine the content of the glucose in the testing solution.

The glucose oxidase used in the oxidase method is usually a biological enzyme. The activity of common biological enzymes is easily affected by the environment, especially affected by the temperature and pH value, the thermal sensitivity is high, and the stability is low. However, the monitoring time of a continuous blood glucose monitor is usually required be 14 days or even longer. During long-term monitoring process, the biological enzymes are affected by too many factors, resulting in fluctuation of blood glucose monitoring data and reduced accuracy, so that further treatment of the diabetes mellitus is not facilitated.

### BRIEF SUMMARY OF THE INVENTION

In order to overcome the defect that existing biological enzymes have low stability in long-term continuous monitoring of blood glucose, the present invention provides a preparation method of a synthetase for continuous monitoring of blood glucose. A glass bead is attached to glucose, and a glucose synthetase with a high polymer structure is formed by a polymerization reaction promoted by an ultraviolet light. Compared with the biological enzymes with protein structures, the high polymer structure is stable and has an obvious stability advantage in heat resistance, pH sensitivity and other aspects. Moreover, ferrocene is added on the glucose synthetase to serve as an electron acceptor, so as to promote the transfer activity of electrons between an induction electrode and the glucose synthetase, thereby improving the accuracy of feedback data of the induction electrode.

The technical solutions adopted by the present invention are as follows.

A preparation method of a synthetase for continuous monitoring of blood glucose is provided. The method includes the preparation steps of
step S1: hydroxylation of a glass bead;
step S2: silanization of the glass bead;
step S3: bonding of the glass bead to glucose;
step S4: synthesis of a polymer particle on a surface of the glass bead to form a glucose synthetase; and
step S5: separation of the highly specific glucose synthetase.

According to the preparation method of a synthetase for continuous monitoring of blood glucose, in step S1, a sodium hydroxide solution and a glass bead are prepared, the glass bead is placed in the sodium hydroxide solution, the sodium hydroxide solution is heated and boiled for a period of time, the glass bead is taken out after the surface is activated, then sodium hydroxide on the surface of the glass bead is removed, and the glass bead is dried.

Further, the content of the sodium hydroxide in the sodium hydroxide solution is 0.8 ml/g.

Further, when the substance on the surface of the glass bead is removed, the glass bead is washed with deionized water for several times first and then washed with an acidic buffer solution.

According to the preparation method of a synthetase for continuous monitoring of blood glucose, in step S2, after 3-isocyanatopropyltriethoxysilane (ICPTES) and N,N-diisopropylethylamine (DIPEA) are dissolved in anhydrous toluene in a ventilation cabinet to treat an inorganic substrate in a mixture, the glass bead obtained in step S1 is added to the mixture for a reaction, taken out after the reaction is completed, and then dried after the unreacted substance on the surface of the glass bead is removed.

Further, the volume fraction of the 3-isocyanatopropyltriethoxysilane (ICPTES) is 5%, and the volume fraction of the N,N-diisopropylethylamine (DIPEA) is 1%.

Further, a 0.5 ml/g 3-isocyanatopropyltriethoxysilane (ICPTES) solution is added to treat the inorganic substrate in the mixture.

Further, the unreacted substance on the surface of the glass bead is removed by washing with an acetone solution.

According to the preparation method of a synthetase for continuous monitoring of blood glucose, in step S3, glucose and N,N-diisopropylethylamine (DIPEA) are dissolved in anhydrous toluene to form a mixed solution, and the glass bead obtained in step S2 is added to the mixed solution, taken out after a reaction is completed, and then dried after the unreacted substance on the surface of the glass bead is removed.

Further, in the mixed solution, the molar concentration of the glucose is 1% of the volume fraction of the 10 mmol/L N,N-diisopropylethylamine (DIPEA).

According to the preparation method of a synthetase for continuous monitoring of blood glucose, in step S4, a polymer for bonding to the glucose on the surface of the glass bead obtained in step S3 is prepared, the polymer and the glass bead are added to a glass reaction vessel, and the glass reaction vessel is irradiated with an ultraviolet light under the protection of an inert gas. Further, pentaerythritol tetra(3-mercaptopropionate) (PETMP), diethyldithiocarbamic acid benzyl ester, α-methacrylic acid (MAA), ethylene glycol dimethacrylate (EGDMA), trimethylolpropane trimethacrylate (TRIM) and ferrocenylmethyl methacrylate (FcMMA) are added to acetonitrile and mixed by shaking.

A polymerization reaction is carried out in the mixed solution under the action of the ultraviolet light. The polymerization reaction carried out under the ultraviolet light has a high reaction rate, more than 90% of substance conversion can be realized within tens of seconds, the reaction rate and the reaction degree can be regulated according to the intensity of the ultraviolet light, and the reaction process is not affected by temperature. In an embodiment, the mixed solution is irradiated only with the ultraviolet light in a dark place, where helium (or an inert gas) is provided for protection to prevent the participation of the air, so as to make the mixed solution polymerized on the surface of the glass bead.

In the prior art, a polymerization reaction with the ultraviolet light is usually used for curing materials, such as curing of coating layers or glue layers, similar to the use of wood products, papers, plastics and other materials that are sensitive to temperature. However, according to the present invention, the ultraviolet light is used for polymerization of the glucose on the surface of the glass bead and the mixed solution. Based on various acrylates which can rapidly absorb energy under the action of the ultraviolet light, a chemical bond is broken to form a free radical, so as to serve as a chain for connecting the glucose on the glass bead and ferrocenylmethyl methacrylate, so that ferrocene is bonded to the glucose synthetase and can replace oxygen as an electron acceptor when reacting with the glucose in the blood glucose. That is to say, the transfer ability of electrons is improved by the ferrocene, and a transfer process of the electrons between the glucose synthetase and an induction electrode is effectively completed, so that measurement data are more accurate, and reactions are more sensitive.

According to the preparation method of a synthetase for continuous monitoring of blood glucose, in step S5, the glass bead and a mixture obtained in step S4 are transferred into an injection tube, and then a supernatant is removed to obtain a highly specific bonded glucose synthetase filtrate.

Further, the process of removing the supernatant includes
step A1: sucking and discarding the supernatant in the injection tube by an SPE tube;
step A2: washing the SPE tube with acetonitrile for several times;
step A3: subjecting the injection tube to incubation in a water bath; and
step A4: repeating step A1 to step A3 for several times.

The present invention implemented by adopting the above solutions has the following beneficial effects. According to the preparation method of a synthetase for continuous monitoring of blood glucose provided by the present invention, the glass bead is bonded to the glucose by activating the surface of the glass bead, and then bonded to a polymer to form the glucose synthetase. The artificial synthetase prepared by the method has low sensitivity to temperature and pH value with a small affected range, and can be directly used in a blood glucose electrochemical sensor.

The glucose synthetase formed by the present invention is a high polymer, while a biological glucose oxidase has a protein structure. Based on a basic chemical theory, the protein has activity, and the high polymer has better thermal stability than the protein structure. Therefore, the glucose synthetase formed by the present invention has higher stability and can adapt to a more complex environment. Similarly, the high polymer has better sensitivity to the pH value than the protein structure, and the glucose oxidase with the protein structure basically only has normal activity in an environment with the pH value close to that of the human body, which is greatly affected by the pH value. Therefore, the glucose synthetase formed by the present invention has higher stability and can adapt to a more complex environment.

### BRIEF DESCRIPTION OF THE DRAWINGS

In order to more clearly illustrate the technical solutions in embodiments of the present invention, drawings required to be used in the embodiments or in the description of the prior art are briefly introduced below. Obviously, the drawings described below are merely some embodiments of the present invention, and other drawings may also be obtained by persons of ordinary skill in the art without creative effort according to these drawings.

FIG. 1 is a synthesis flow diagram of bonding of a glass bead to glucose in the present invention.

FIG. 2 is a comparison diagram of the pH sensitivity of a glucose synthetase in the present invention and a glucose oxidase (biological enzyme).

FIG. 3 is a comparison diagram of the thermal stability of a glucose synthetase in the present invention and a glucose oxidase (biological enzyme).

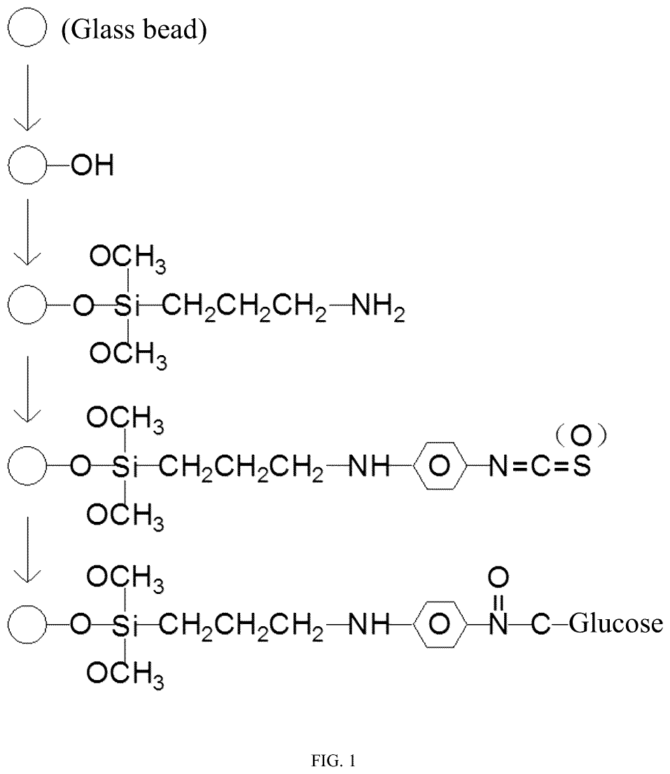

### DETAILED DESCRIPTION OF THE INVENTION

In order to make the technical problems to be solved, technical solutions and beneficial effects of the present invention clearer and more understood, the present invention is further explained in detail below in combination with the attached drawings and embodiments. It should be understood that the specific embodiments described herein are only for illustrating but not for limiting the present invention.

According to a preparation method of a synthetase for continuous monitoring of blood glucose, a glass bead, used as a reaction “vessel”, is subjected to hydroxylation and silanization, followed by bonding to glucose. Then, a polymer particle is chemically synthesized to undergo a reaction with the glass bead, and a finally resulting substance is separated in a water bath to form a highly specific bonded glucose synthetase.

Step S1: Hydroxylation of a glass bead:

60 grams of a glass bead and a 1 molar concentration of a sodium hydroxide solution were prepared, where the content of sodium hydroxide in the sodium hydroxide solution was 0.8 ml/g. The glass bead was placed in the sodium hydroxide solution, and the sodium hydroxide solution was heated and boiled for 15 minutes for activating the surface of the glass bead. Then, the glass bead was taken out, the surface was washed with deionized water with 200 ml of deionized water each time, and then the sodium hydroxide on the surface of the glass bead was neutralized with a phosphoric acid buffer solution to ensure that the sodium hydroxide was completely removed. Then, the glass bead was dried in an oven for 3 hours, where the temperature of the oven was set as 80° C.

Step S2: Silanization of the glass bead:

3-isocyanatopropyltriethoxysilane (ICPTES) with a volume fraction of 5% and N,N-diisopropylethylamine (DIPEA) with a volume fraction of 1% were prepared and dissolved in anhydrous toluene in a ventilation cabinet, 3-isocyanatopropyltriethoxysilane (ICPTES) with a content of 0.5 ml/g was used to treat an inorganic substrate, and then the glass bead was added to induce a reaction of a mixture at a temperature of 50° C. for 18 hours. The glass bead was taken out after the reaction was completed, washed with an acetone solution for two times, and then dried in an oven for 3 hours, where the temperature of the oven was set as 80° C.

Step S3: Bonding of the glass bead to glucose:

Glucose was prepared, and the glucose and N,N-diisopropylethylamine (DIPEA) were dissolved in anhydrous toluene to form a mixed solution, where the molar concentration of the glucose was 1% of the volume fraction of the 10 mmol/L N,N-diisopropylethylamine (DIPEA). The glass bead was put into the mixed solution for mixing to carry out a reaction of the two substances at a temperature of 50° C. for 18 hours. After the reaction was completed, the glass bead was taken out, washed with acetone for two times, and then dried in an oven for 3 hours, where the temperature of the oven was set as 60° C.

Step S4: Synthesis of a polymer particle on the surface of the glass bead to form a glucose synthetase:

0.18 g of pentaerythritol tetra(3-mercaptopropionate) (PETMP) with a molar concentration of 0.37 mmol/L, 0.75 g of diethyldithiocarbamic acid benzyl ester with a molar concentration of 3.14 mmol/L, 2.88 g of α-methacrylic acid (MAA) with a molar concentration of 33.47 mmol/L, 3.24 g of ethylene glycol dimethacrylate (EGDMA) with a molar concentration of 16.35 mmol/L, 3.24 g of trimethylolpropane trimethacrylate (TRIM) with a molar concentration of 9.57 mmol/L and 1.5 g of ferrocenylmethyl methacrylate (FcMMA) with a molar concentration of 3.347 mmol/L were separately weighed and added to a 10 ml glass flask, and 10.52 g of acetonitrile was added to the glass flask and mixed by shaking.

A 200 ml glass vessel was prepared, 30 g of the glass bead was put into the glass vessel, and the above mixed solution was poured into the glass vessel. Then, the glass vessel, the outside of which was protected by helium, was irradiated in an irradiation range of an ultraviolet lamp for 5 minutes, and a polymerization reaction of the mixed solution was induced by an ultraviolet light. In this embodiment, the ultraviolet light is provided by a synthesis device, and the synthesis device is a sealed black box with a lid. The inside of the sealed black box is provided with four ultraviolet light tubes and a glass culture dish with a lid, where common ultraviolet light tubes are used, and the power of each ultraviolet light tube is 15 W. The sealed black box is capable of blocking the ultraviolet light completely to prevent overflow. Among the four ultraviolet light tubes, two ultraviolet light tubes are arranged side by side at a distance that enables stable placement of the glass culture dish, and the width of the two ultraviolet light tubes is similar to the diameter of the round glass culture dish, which is at least 80% of the diameter; and the other two ultraviolet light tubes are arranged on two sides of the round glass culture dish and are close to the glass culture dish, and the tops of the other two ultraviolet light tubes are higher than the two ultraviolet light tubes used for supporting the glass culture dish, so that the glass culture dish is limited by the four ultraviolet light tubes, and a groove for placing the glass culture dish is formed by the four ultraviolet light tubes. A switch, a light intensity control device a timing device and other control devices of the ultraviolet light tubes are all arranged outside through the sealed black box, so as to control the switching state and working time of the ultraviolet light tubes by manual operation. Step S5: Separation of the highly specific glucose synthetase:

All substances in the glass vessel, including the glass bead and the mixed solution, were transferred into an injection tube. Extraction and separation were conducted with an SPE tube to remove an undesired supernatant, including an unreacted monomer and a polymer with low affinity. The supernatant was sucked and discarded by the SPE tube first, then the SPE tube was washed with acetonitrile for 10 times, and the injection tube was transferred to a water bath for incubation for 15 minutes at a temperature of 60° C. In this way, the above operations were repeated for 3 times to finally collect a highly specific bonded glucose synthetase filtrate in the injection tube, and then the highly specific bonded glucose synthetase filtrate was cooled to 4° C. for storage. The obtained product can be used after dialysis and purification.

Material components and corresponding dosages used in this embodiment are as shown in the following table.

| Component | Use amount |
|---|---|
| α-methacrylic acid (MAA) | 2.88 g (33.47 mmol) |
| Ethylene glycol dimethacrylate (EGDMA) | 3.24 g (16.35 mmol) |
| Trimethylolpropane trimethacrylate (TRIM) | 3.24 g (9.57 mmol) |
| Diethyldithiocarbamic acid benzyl ester | 0.75 g (4.17 mmol) |
| Pentaerythritol tetra(3-mercaptopropionate) | 0.18 g (0.4 mmol) |
| (PETMP) | |
| Ferrocenylmethyl methacrylate (FcMMA) | 0.25 g (0.875 mmol) |
| Acetonitrile | 11.52 g |

The glucose synthetase prepared by the present invention is mainly different from a glucose oxidase in structure. The glucose synthetase of the present invention is a high polymer obtained by a polymerization reaction promoted by the ultraviolet light, while the glucose oxidase is a substance with a protein structure derived from a biological body. Therefore, based on structural properties, the protein structure has higher requirements for a living environment, poor stability and higher requirements for the external environment, while the high polymer has no such problems and can maintain the activity in more environments.

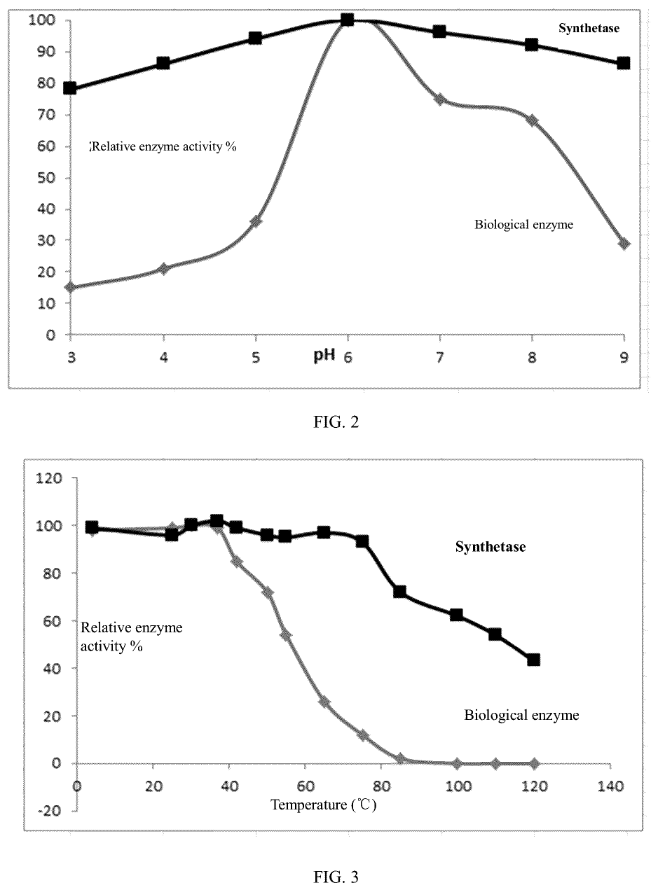

As shown in FIG. 2 and FIG. 3, the glucose synthetase of the present invention has better properties than the glucose oxidase in terms of pH sensitivity and thermal stability, and has a much higher adaptive range than the glucose oxidase. The glucose synthetase can still maintain high enzyme activity in an environment with a high pH value or a low pH value, while the activity of the glucose oxidase can be maintained only in an environment with a pH value close to that of the human body and is greatly reduced at other pH values. In terms of heat resistance, the glucose synthetase of the present invention can maintain relatively stable high activity until the temperature reaches about 80° C. and still has certain activity at a high temperature of 120° C., while the activity of the glucose oxidase is obviously decreased at a temperature higher than about 37° C. of the human body and is decreased sharply, and when the temperature reaches about 90° C., the glucose oxidase has almost no activity, which cannot achieve any effect.

The foregoing descriptions are merely preferred exemplary embodiments of the present invention and are not intended to be limitations of the present invention, and any modifications, equivalent substitutions, improvements and the like made within the spirit and principles of the present invention are intended to be embraced by the protection range of the present invention.

What is claimed is:

1. A preparation method of a synthetase for continuous monitoring of blood glucose, wherein the method comprises the preparation steps of
step S1: hydroxylation of a glass bead;
step S2: silanization of the glass bead;
step S3: bonding of the glass bead to glucose;
step S4: synthesis of a polymer particle on a surface of the glass bead to form a glucose synthetase; and
step S5: separation of the highly specific glucose synthetase.

2. The preparation method of a synthetase for continuous monitoring of blood glucose according to claim 1, wherein in step S1, a sodium hydroxide solution and a glass bead are prepared, the glass bead is placed in the sodium hydroxide solution, the sodium hydroxide solution is heated and boiled for a period of time, the glass bead is taken out after the surface is activated, then sodium hydroxide on the surface of the glass bead is removed, and the glass bead is dried.

3. The preparation method of a synthetase for continuous monitoring of blood glucose according to claim 2, wherein when the substance on the surface of the glass bead is removed, the glass bead is washed with deionized water for several times first and then washed with an acidic buffer solution.

4. The preparation method of a synthetase for continuous monitoring of blood glucose according to claim 1, wherein in step S2, after 3-isocyanatopropyltriethoxysilane (ICPTES) and N,N-diisopropylethylamine (DIPEA) are dissolved in anhydrous toluene in a ventilation cabinet to treat an inorganic substrate in a mixture, the glass bead obtained in step S1 is added to the mixture for a reaction, taken out after the reaction is completed, and then dried after the unreacted substance on the surface of the glass bead is removed.

5. The preparation method of a synthetase for continuous monitoring of blood glucose according to claim 4, wherein the volume fraction of the 3-isocyanatopropyltriethoxysilane (ICPTES) is 5%, and the volume fraction of the N,N-diisopropylethylamine (DIPEA) is 1%.

6. The preparation method of a synthetase for continuous monitoring of blood glucose according to claim 5, wherein a 0.5 ml/g 3-isocyanatopropyltriethoxysilane (ICPTES) solution is added to treat the inorganic substrate in the mixture.

7. The preparation method of a synthetase for continuous monitoring of blood glucose according to claim 1, wherein in step S3, glucose and N,N-diisopropylethylamine (DIPEA) are dissolved in anhydrous toluene to form a mixed solution, and the glass bead obtained in step S2 is added to the mixed solution, taken out after a reaction is completed, and then dried after the unreacted substance on the surface of the glass bead is removed.

8. The preparation method of a synthetase for continuous monitoring of blood glucose according to claim 1, wherein in step S4, a polymer for bonding to the glucose on the surface of the glass bead obtained in step S3 is prepared, the polymer and the glass bead are added to a glass reaction vessel, and the glass reaction vessel is irradiated with an ultraviolet light under the protection of an inert gas.

9. The preparation method of a synthetase for continuous monitoring of blood glucose according to claim 8, wherein pentaerythritol tetra(3-mercaptopropionate) (PETMP), diethyldithiocarbamic acid benzyl ester, α-methacrylic acid (MAA), ethylene glycol dimethacrylate (EGDMA), trimethylolpropane trimethacrylate (TRIM) and ferrocenylmethyl methacrylate (FcMMA) are added to acetonitrile and mixed by shaking.

10. The preparation method of a synthetase for continuous monitoring of blood glucose according to claim 1, wherein in step S5, the glass bead and a mixture obtained in step S4 are transferred into an injection tube, and then a supernatant is removed to obtain a highly specific bonded glucose synthetase filtrate.
